(19) United States
(12) Patent Application Publication
(10) Pub. No.: US 2007/0233113 A1
(43) Pub. Date: Oct. 4, 2007

(54) Condylar plate

(75) Inventors: Michael Kaelblein, Westfield, NJ (US); John Minier, Highland Mills, NY (US); Mari Truman, Warsaw, IN (US)

Correspondence Address: MORGAN, LEWIS & BOCKIUS LLP, 1701 MARKET STREET, PHILADELPHIA, PA, 19103-2921, US

(73) Assignee: Small Bone Innovations, Inc.

(21) Appl. No.: 11/523,129

(22) Filed: Sep. 18, 2006

Related U.S. Application Data
(60) Provisional application No. 60/718,136, filed on Sep. 16, 2005.

(51) Int. Cl. A61F 2/30

(52) U.S. Cl. 606/69

(57) ABSTRACT

A condylar plate having at least two tines and useful for small bone surgery is disclosed. A method for implanting the plate is also disclosed.

### CROSS REFERENCE TO RELATED APPLICATIONS

This application claims priority to U.S. Provisional Patent Application No. 60/718,136, filed on Sep. 16, 2005, which is herein incorporated by reference in its entirety.

### SUMMARY OF INVENTION

The present invention relates to a bone implant, in particular, a condylar plate for fixation of a small bone fracture. In one embodiment of the present invention, the condylar plate of the present invention includes two or more tines connected to a plate having one or more apertures configured to receive a fastener. In one embodiment, the tines of the condylar plate are configured to be fixed to the condyle portion of a small bone.

In another embodiment of the present invention, the condylar plate includes scalloped edges adjacent each of said apertures.

In yet another embodiment of the present invention, the tines on the condylar plate are normal to the plate. In another embodiment, all or a portion of the tines are removable. In another embodiment, the tines are flexible. In an embodiment of the invention, the tines are flexible from about 0 to about 90 degrees. In another embodiment, the tines are flexible from about 0 to 45 degrees.

In an embodiment of the invention, a method of fitting a condylar plate to a small bone in a patient is disclosed. In one embodiment, the method includes molding a pliable condylar plate template to the small bone of a patient, drilling holes in the bone through apertures in the template at the location where the tines will be fixated to the bone, and conforming (e.g., bending) the condylar plate to a configuration that corresponds to the template configuration. In one embodiment, the template is substantially identical to the condylar plate, except that it has apertures where the tines would be located.

### BRIEF DESCRIPTION OF THE DRAWINGS

The foregoing summary, as well as the following detailed description of various embodiments of the invention, will be better understood when read in conjunction with the appended drawings. Drawings are provided for the purpose of illustrating certain embodiments of the invention. It should be understood, however, that the invention is not limited to the precise arrangements and instrumentalities shown, including the recited dimensions. In the drawings:

FIG. 1 illustrates an isometric view of a condylar plate according to the present invention.

FIG. 2 illustrates a cross-sectional view of a longitudinal section of a condylar plate along lines A-A in FIG. 1, according to the present invention.

FIG. 3, comprising FIGS. 3A-3E, illustrate different views of a condylar plate according to the present invention. FIG. 3A illustrates an isometric view. FIG. 3B illustrates a front view of the ends of the tines. FIG. 3C is a top view. FIG. 3D is a side view. FIG. 3E is a cross-sectional side view.

FIG. 4, comprising FIGS. 4A-4C, illustrate different view of a condylar plate according to the present invention. FIG. 4A illustrates a top view, with tines stretched to a position parallel to the longitudinal axis of a condylar plate according to the present invention. FIG. 4B is a side view. FIG. 4C is a cross sectional view of cut D-D in FIG. 4B.

FIG. 5, comprising FIGS. 5A-5M, illustrates a preferred embodiment of a condylar plate according to the present invention. FIG. 5A is a detailed illustration of portion A of FIG. 5B. FIG. 5B illustrates a front view of a condylar plate according to the present invention. FIG. 5C illustrates a side view of a condylar plate according to the present invention. FIG. 5E is a 6:1 scaled view of cross section B-B in FIG. 5G. FIG. 5F is an isometric view of a condylar plate according to the present invention. FIG. 5G is a top view of a condylar plate according to the present invention. FIG. 5H is another top view of a condylar plate according to the present invention. FIG. 5I is an 8:1 scaled view of cross section D-D in FIG. 5L. FIG. 5J is an 8:1 scaled view of cross section E-E in FIG. 5L. FIG. 5K is a top view of a condylar plate according to the present invention. FIG. 5L is a side view of a condylar plate according to the present invention. FIG. 5M is a top view of a condylar plate according to the present invention, with straightened tines.

FIG. 6, comprising FIGS. 6A-6M, illustrates a preferred embodiment of a condylar plate according to the present invention. FIG. 6A is a detailed illustration of portion A of FIG. 6B. FIG. 6B illustrates a front view of a condylar plate according to the present invention. FIG. 6C illustrates a side view of a condylar plate according to the present invention. FIG. 6E is a 6:1 scaled view of cross section B-B in FIG. 6G. FIG. 6F is an isometric view of a condylar plate according to the present invention. FIG. 6G is a top view of a condylar plate according to the present invention. FIG. 6H is another top view of a condylar plate according to the present invention. FIG. 6I is an 8:1 scaled view of cross section D-D in FIG. 6L. FIG. 6J is an 8:1 scaled view of cross section E-E in FIG. 6L. FIG. 6K is a top view of a condylar plate according to the present invention. FIG. 6L is a side view of a condylar plate according to the present invention. FIG. 6M is a top view of a condylar plate according to the present invention, with straightened tines.

FIG. 7, comprising FIGS. 7A-7M, illustrates a preferred embodiment of a condylar plate according to the present invention. FIG. 7A is a detailed illustration of portion A of FIG. 7B. FIG. 7B illustrates a front view of a condylar plate according to the present invention. FIG. 7C illustrates a side view of a condylar plate according to the present invention. FIG. 7E is a 6:1 scaled view of cross section B-B in FIG. 7G. FIG. 7F is an isometric view of a condylar plate according to the present invention. FIG. 7G is a top view of a condylar plate according to the present invention. FIG. 7H is another top view of a condylar plate according to the present invention. FIG. 7I is an 8:1 scaled view of cross section D-D in FIG. 7L. FIG. 7J is an 8:1 scaled view of cross section E-E in FIG. 7L. FIG. 7K is a top view of a condylar plate according to the present invention. FIG. 7L is a side view of a condylar plate according to the present invention. FIG. 7M is a top view of a condylar plate according to the present invention, with straightened tines.

FIG. 8 illustrates the cutting angle for a tine when shortening the tine of a condylar plate according to the present invention.

FIG. 9 illustrates the cutting angle for a tine when removing the tine from a condylar plate according to the present invention.

### DETAILED DESCRIPTION

The present invention is related to bone plates, in particular, a condylar plate and methods for fitting and using bone plates.

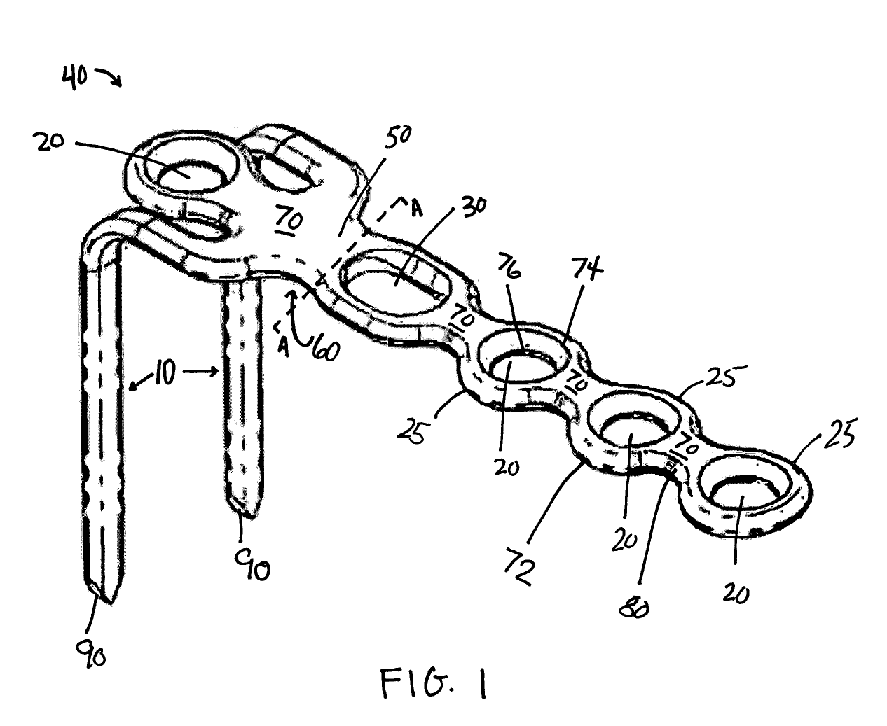

Referring to FIG. 1, in an embodiment of the present invention, the condylar plate 40 has a longitudinal section 70 with apertures 20, 30, and at least two tines 10 having a beveled edge 90 perpendicular to longitudinal section 70. Longitudinal section 70 has a top profile 50, a bottom profile 60, and two side profiles 80, side profiles 80 being substantially identical. In an embodiment of the invention, condylar plate 40 is manufactured in different sizes to accommodate different sized anatomy, such as child, adolescent, and adult anatomy.

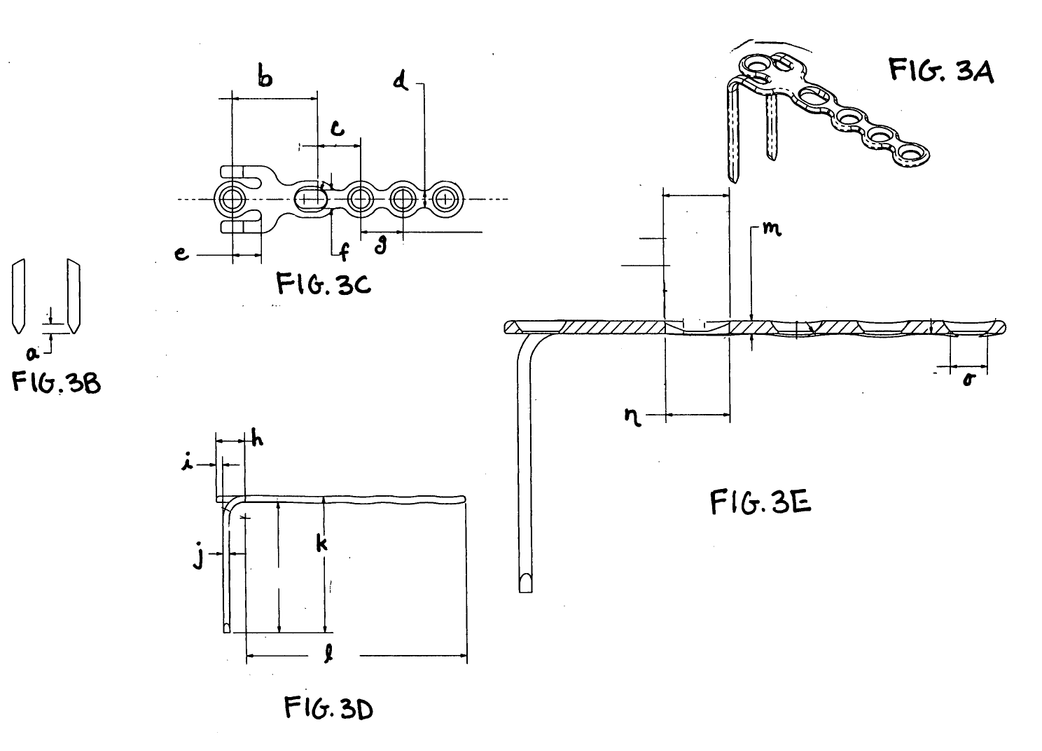

Referring to FIG. 3D, in an embodiment of the invention, the length (l+h) of longitudinal section 70 of condylar plate 40 according to the present invention is from about 15 millimeters to about 40 millimeters, from end to end. In another embodiment, the length (l+h) of longitudinal section 70 is from about 20 millimeters to about 35 millimeters. In another embodiment, the length (l+h) of longitudinal section 70 is from about 23.4 millimeters to about 37.6 millimeters.

In an embodiment of the invention, condylar plate 40 is a low-profile plate. Referring to FIG. 3E, in an embodiment of the invention, the thickness (m) of condylar plate 40 is from about 0.4 millimeter to about 1.2 millimeters. In another embodiment, the thickness (m) of plate 40 is from about 0.5 to about 1.05 millimeters. In another embodiment, the thickness (m) of plate 40 is from about 0.575 to about 1.0 millimeter.

In an embodiment of the present invention, condylar plate 40 includes two or more tines 10 configured to be fixed to the condyle portion of a small bone. One advantage to using at least two tines is that optimal stability is increased. In an embodiment of the present invention, tines 10 are substantially normal to longitudinal portion 70 of condylar plate 40 according to the present invention. In an embodiment of the invention, tines 10 have a beveled edge 90 for facilitating insertion of tine 10 into a condyle of a bone. Referring to FIG. 3B, in one embodiment, the bevel depth (a) of bevel 90 is from about 0.5 to about 1 millimeter. In another embodiment the bevel depth (a) of bevel 90 is from about 0.7 to about 0.9 millimeter. In another embodiment, the bevel depth (a) of bevel 90 is about 0.8 millimeter. In an embodiment of the invention, each tine 10 is beveled at about a 60 degree angle.

Referring to FIG. 3D, in an embodiment of the present invention, the length (k) of tines 10 is about 8 to about 20 millimeters long, taking into account the thickness of the plate. In another embodiment, the length (k) of tines 10 is about 10 to about 16 millimeters long, taking into account the thickness of the plate. In another embodiment, the length (k) of tines 10 is about 12 millimeters long, taking into account the thickness of the plate.

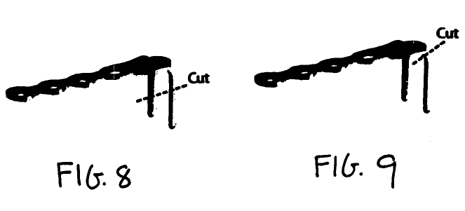

In an embodiment of the invention, tines 10 are fully or partially removable. That is, tines 10 can be clipped (e.g., with side cutter pliers or other appropriate cutting device) to a particular length to fit an individual bone, as shown in FIG. 8, by cutting tine 10 substantially parallel to longitudinal axis 70 of plate 40 according to the present invention. In an embodiment of the invention, if tine 10 is not needed, it can be clipped off completely, as shown in FIG. 9, by cutting parallel to plate 40 to preserve the beveled upper corner of tine 10.

In an embodiment of the present invention, condylar plate 40 has two tines 10 that are spaced from about 3 to about 9 millimeters apart. In another embodiment, tines 10 are spaced about 3.5 to about 8.5 millimeters apart. In another embodiment, tines 10 are spaced about 4 to about 6.4 millimeters apart.

In an embodiment of the invention, the thickness (j) of each of tines 10 present on condylar plate 40 is about 0.5 millimeter to about 2 millimeters. In another embodiment, the thickness (j) of each of tines 10 is about 0.7 to about 1.5 millimeters. In another embodiment, the thickness (j) of each of tines 10 is about 1 to about 1.3 millimeters.

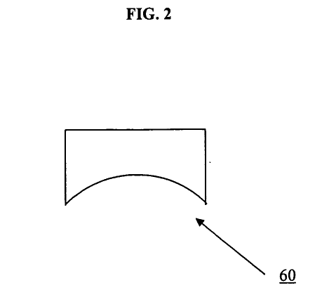

In an embodiment of the present invention, longitudinal section 70 of condylar plate 40 is flat. In another embodiment, condylar plate 40, including tines 10, is flexible. That is, plate 40 can be conformed (e.g., by bending) to a configuration that corresponds to and fits the anatomy (e.g., to the contour of a bone or bones on which it is placed). In another embodiment, plate 40 is conformed to achieve optimal fixation. Referring to FIG. 2, in an embodiment of the invention, bottom profile 60 of longitudinal section 70 of condylar plate 40 is slightly curved. In one embodiment, bottom profile 60 has a radius of curvature of about 0 to about 20 millimeters. In another embodiment, bottom profile 60 has a radius of curvature of about 5 to 10 millimeters. In another embodiment, bottom profile 60 has a radius of curvature of about 8.5 millimeters. In another embodiment, bottom profile 60 has a radius of curvature of about 7 millimeters. One advantage to curving bottom profile 60 is that plate 40 more easily conforms to human anatomy (e.g., shape of a bone) to provide optimal fixation.

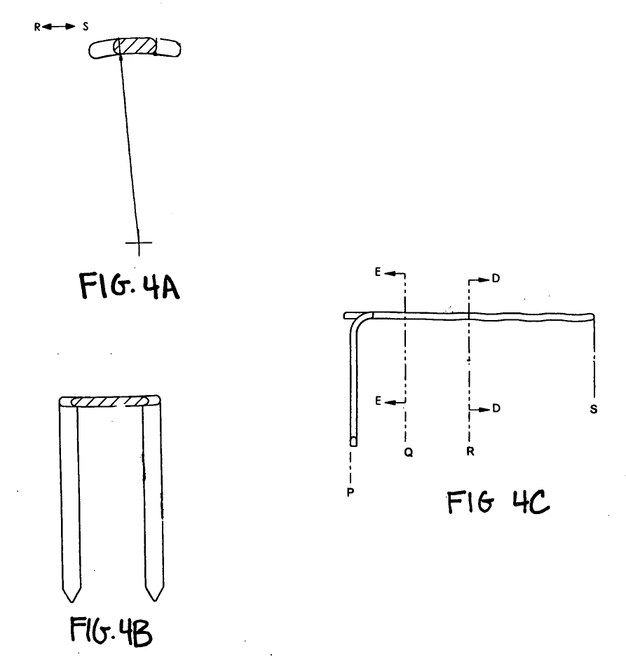

In an embodiment of the invention, longitudinal section 70 of plate 40 transitions from a flat to a radiused cross-section. Referring to FIGS. 4A, 4B, and 4C, in the region that extends from P to Q, the cross section is essentially flat (see cross section of E-E in FIG. 4B). In the region that extends from Q to R, the transition from flat to radiused occurs. In the region that extends from R to S, the cross section is curved (see cross section D-D in FIG. 4A).

In another embodiment of the invention, condylar plate 40 includes two or more apertures 20, 30 configured to receive a fastener. In an embodiment of the invention, apertures 20 are substantially rounded and cup-shaped, and are defined by a Gage Ball having a specific diameter. In an embodiment of the invention, apertures 20 are defined by a Gage Ball having a diameter of from about 2.0 millimeters to about 5 millimeters. In another embodiment, the Gage Ball diameter is from about 2.3 millimeters to about 4.5 millimeters. In another embodiment, the Gage Ball diameter is about 2.6 millimeters.

In an embodiment of the invention, apertures 20, 30 are tapered from top profile 50 of plate 40 to bottom profile 60 of plate 40, such that upon insertion of a fastener, the fastener head will be substantially flush with top profile 50 of plate 40. One advantage of having the fasteners flush or substantially flush with top profile 50 of plate 40 is that it plays a role in minimizing soft tissue irritation.

Referring to FIG. 1, in an embodiment of the invention, apertures 20 have two diameters 72, 74 at top profile 50 of condylar plate 40, and one diameter 76 at bottom profile 60. In an embodiment of the invention, the top profile outer diameter 72 is from about 2 to about 5 millimeters. In another embodiment, top profile outer diameter 72 is from about 2.5 to about 4.5 millimeters. In another embodiment, top profile outer diameter 72 is from about 3.45 to about 4.366 millimeters.

In an embodiment of the invention, the top profile inner diameter 74 is from about 2.5 millimeters to about 5.5 millimeters. In another embodiment, top profile inner diameter 74 is from about 3 millimeters to about 4.725 millimeters.

In another embodiment of the invention, the bottom profile diameter 76 (identified as o in FIG. 3E) of each aperture 20 is from about 1 millimeter to about 3.5 millimeters. In another embodiment, bottom profile diameter 76 of each aperture is from about 1.5 millimeters to about 3 millimeters. In another embodiment, bottom profile diameter 76 is from about 1.725 millimeters to about 2.625 millimeters.

Referring to FIG. 3C, in an embodiment of the invention, at least two of apertures 20 are spaced substantially equal apart, and the distance (g) between the centers of the two apertures 20 is from about 3 to about 8 millimeters. In another embodiment, the distance (g) is from about 4 to about 7 millimeters. In another embodiment, the distance (g) is from about 4 to 6.5 millimeters.

Referring to FIG. 1, in an embodiment of the invention, an aperture 20 is located at a position between tines 10. In one embodiment, tines 10 connect to condylar plate 40 at a point 15 behind one or more apertures 20. In another embodiment, tines 10 are integral with condylar plate 40. In an embodiment, aperture 20 is located between the tines, and can be bent at an angle to longitudinal section 70 of plate 40 for optimal fixation. In one embodiment, tines 10 bend downward at a substantially 90 degree angle.

Referring to FIG. 3C, in an embodiment of the present invention, the distance (e) between the center point of aperture 20 between tines 10 and the beginning point 15 of tines 10 is from about 2.0 millimeters to about 5.0 millimeters. In an embodiment of the present invention, the distance (e) between the center point of aperture 20 between tines 10 and the beginning point 15 of tines 10 is from about 2.4 millimeters to about 4.2. In an embodiment of the present invention, the distance (e) between the center point of aperture 20 between tines 10 and the beginning point 15 of tines 10 is from about 2.7 millimeters to about 3.9 millimeters.

Referring to FIG. 3D, in an embodiment of the invention, aperture 20 located between the tines extends a distance (i) from tines 10. In one embodiment, distance (i) is from about 0.4 millimeters to about 1.6 millimeters. In another embodiment, distance (i) is from about 0.5 millimeters to about 1.5 millimeters. In another embodiment, distance (i) is from about 0.604 millimeters to about 1.32 millimeters.

In another embodiment of the invention, at least one aperture 30 is configured to provide up to 1 millimeter of movement during placement, such movement being in at least a one-axis direction to ensure optimal alignment of condylar plate 40 with a bone. Referring to FIG. 3E, in an embodiment of the invention, aperture 30 is oval and cup-shaped and has a length (n) along longitudinal axis 70 of condylar plate 40 of about 2 to about 7 millimeters. In another embodiment, the length (n) is from about 2.5 to about 6 millimeters. In another embodiment, the length (n) is from about 3.4 to about 5.6 millimeters. In an embodiment of the invention, aperture 30 has a bottom profile width (f) of from about 1.0 millimeter to about 3 millimeters. In another embodiment of the invention, aperture 30 has a bottom profile width (f) of from about 1.3 millimeter to about 2.5 millimeters. In another embodiment of the invention, aperture 30 has a bottom profile width (f) of from about 1.725 millimeter to about 2.225 millimeters.

In an embodiment of the invention, aperture 30 has two radii, a left radius, closer to tines 10, and a right radius, closer to the other apertures, which radii can be the same or different, as shown in FIG. 3C. Referring to FIG. 3C, in an embodiment of the invention, the distance (b) of between the center of aperture 20 between tines 10 and the right radius is about 6.000 millimeters to about 14.000 millimeters. In another embodiment, the distance (c) from the right radius to the center of the next aperture 20 is from about 7.000 millimeters to about 12.000 millimeters. In another embodiment, the distance (c) from the right radius to the center of the next aperture 20 is from about 8.000 millimeters to about 11.000 millimeters.

In an embodiment of the invention, the edges 25 of plate 40 are sculpted, for example, scalloped. One advantage of sculpting edges 25 of plate 40 is that it simplifies the contouring of plate 40, and also reduces the aperture stress concentration, thus apertures 20, 30 better resist fatigue. Another advantage of sculpting edges 25 of plate 40 is that it aids in optimizing vascularization around plate 40, which is important to the healing process. Another advantage is that sculpted edges 25 of plate 40 improve the fit of plate 40, thereby optimizing stability of plate 40 after implantation, and protection of soft tissue. Referring to FIG. 3C, in an embodiment of the invention, plate 40 is sculpted such that the shortest width (d) of plate 40 between apertures 20, 30 is from about 1.000 millimeter to about 3.000 millimeters. In another embodiment of the invention, plate 40 is sculpted such that the shortest width (d) of plate 40 between apertures 20, 30 is from about 1.300 millimeters to about 2.500 millimeters. In another embodiment of the invention, plate 40 is sculpted such that the shortest width (d) of plate 40 between apertures 20, 30 is from about 1.6 millimeters to about 2.200 millimeters.

In an embodiment of the invention, fasteners useful to secure or attach plate 40 to a bone include, for example, screws, k-wires, rivets, pins, and combinations thereof. In certain embodiments, plate 40 includes one or more apertures 20, 30 to receive or accommodate the selected fastener (e.g., screws, k-wires, rivets, and/or pins). In an embodiment of the invention, condylar plate 40 is configured to accommodate 1.5 millimeter bone screws. In another embodiment, condylar plate 40 is configured to accommodate 2.0 millimeter bone screws. In another embodiment, condylar plate 40 is configured to accommodate 2.4 millimeter bone screws.

In an embodiment of the invention, plate 40 is constructed from a medical grade biocompatible material (e.g., metal, alloy, polymer, cobalt-chrome alloys, for example MP35N, BioDur™, and 22-13-5). In one embodiment, the metal is stainless steel (e.g., polished stainless steel). In an embodiment of the invention, the stainless steel is cold-worked. In one embodiment, the cold-worked stainless steel provides added strength to the plate over titanium or other alloys. One advantage of using stainless steel is that it reduces tendon adhesion in dorsal plating and does not appear to cause a severe inflammatory response. In another embodiment, the plate is constructed from titanium. In another embodiment, the plate is constructed from TAV titanium alloy.

Referring again to FIG. 1, in an embodiment of the invention, a condylar plate 40 includes two tines 10. In one embodiment, tines 10 are configured to be fixed to the condyle portion of a small bone, for example, a bone in the finger. In one embodiment, condylar plate 40 also includes at least one aperture configured to receive a fastener. In another embodiment, condylar plate 40 includes two to five apertures 20, 30 configured to receive fasteners. In another embodiment, condylar plate 40 includes five apertures 20, 30 configured to receive fasteners. In one embodiment, aperture 30 is configured to provide up to 1 millimeter of movement in a one-axis direction to ensure a good fit of condylar plate 10 to the bone. In one embodiment, apertures 20, 30 are wider on the top profile 50 of condylar plate 40 than on the bottom profile 60 such that when fasteners are introduced into apertures 20, 30, the head of the fasteners will be substantially flush with top profile 50. In an embodiment of the invention, bottom profile 60 is slightly curved to match or contour with the shape of the bone (see FIG. 2). In an embodiment of the invention, bottom profile 60 has a radius of curvature of about 5 to 10 millimeters. In another embodiment, the radius of curvature is about 8.5 millimeters. This curvature increases the strength of condylar plate 40, allows condylar plate 40 to fit the bone axially, and reduces slippage of condylar plate 40 from the bone when drilling or inserting fasteners. In addition, the curvature keeps condylar plate 40 at a lower profile, thereby reducing soft tissue irritation upon the soft tissue closing over plate 40.

The present invention also discloses a method for fitting a condylar plate to a bone.

In an embodiment of the invention, condylar plate 40 is fitted to a bone using a template. In one embodiment, the template is substantially identical in size and shape to condylar plate 40, except that no tines are present. Instead, the template includes small apertures in a location substantially identical to where tines are present on the condylar plate. The apertures on the template are used to drill holes in the bone where the tines will be fixed.

In an embodiment of the invention, the template is constructed from titanium. One advantage of titanium is that it can be color-coded. In an embodiment of the invention, the template includes apertures for k-wires. In an embodiment of the invention, the template is constructed of a pliable material. In an embodiment of the invention, the template is temporarily fixed to the bone using a k-wire. In another embodiment, the template is temporarily fixed to the bone using a clamp. Once fixed, the template is molded around the bone at a position where the condylar plate will be fixed. Holes are drilled into the bone through the small apertures on the template at the location where the tines on the condylar plate will be fixed to the bone. The template is then removed, and the condylar plate is bent into shape with, for example, bending pliers, to conform the plate to the shape of the template. One advantage of using a template is that it avoids the surgeon having to return to the fracture site multiple times to ensure the correct fit for the plate.

### EXAMPLES

### Example 1

### 1.5 Millimeter Condylar Plate

The 1.5 millimeter condylar plate 40 is configured with five apertures 20, 30, all of which are configured to receive a 1.5 millimeter bone screw. Four of apertures 20 are substantially rounded and cup-shaped, each having a diameter defined by a Gage Ball having about a 2.60 millimeter diameter. Apertures 20, 30 are situated along longitudinal axis 70 in a substantially straight line as follows: There is a substantially round aperture 20 located at the top of plate 40 between tines 10 which is bendable to optimize fixation of plate 40 to the bone. Aperture 30 is located just below tines 10 and is oval shaped and has a length along longitudinal axis 70 of plate 40 of about 3.000 millimeters. Aperture 30 is configured to receive the 1.5 millimeter screw, but allows for about 1 millimeter of movement in longitudinal axis 70 of plate 40 to optimize the fixation of plate 40 on the bone. There are three substantially round apertures 20 that follow the oval-shaped aperture 30, which are spaced substantially equally apart at a distance of about 4,000 millimeters from center point to center point of each aperture 20.

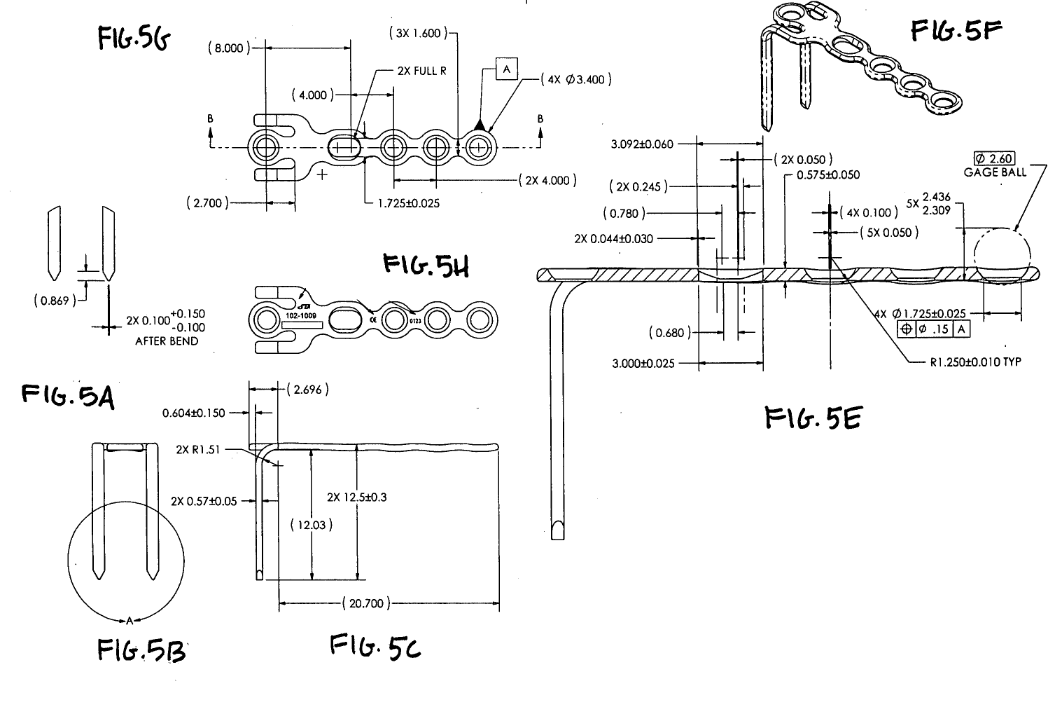

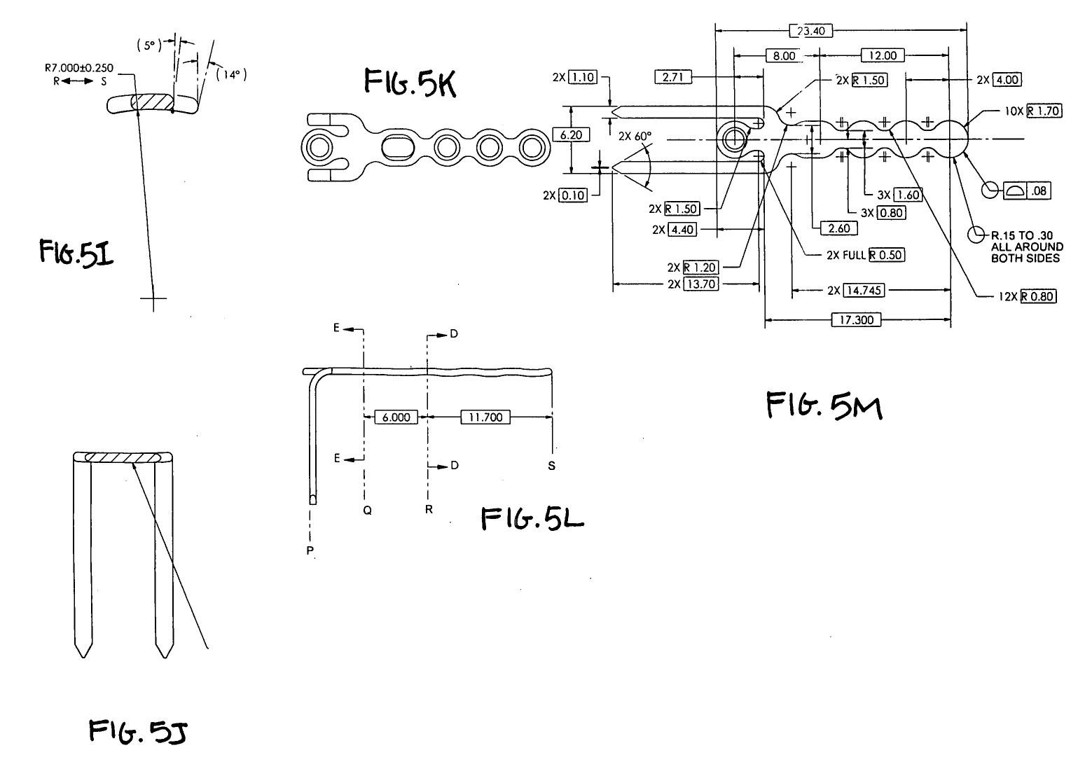

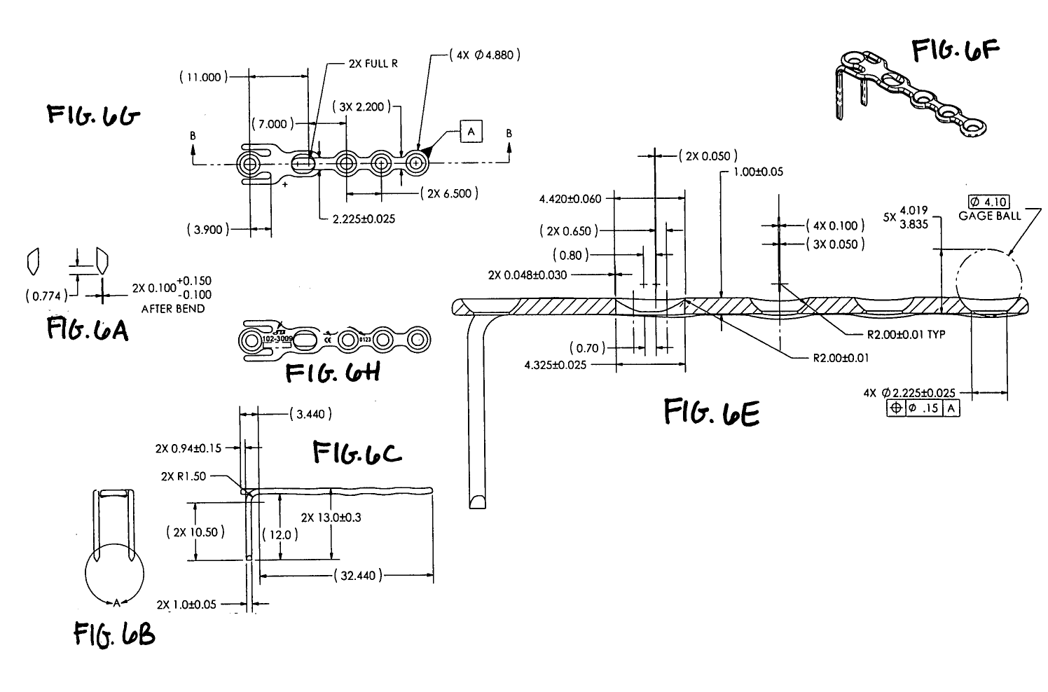

Plate 40 is about 23,396 millimeters long and about 0.575 millimeters thick. Tines 10 are about 12.5 millimeters long, taking into account the thickness of plate 40. Tines 10 are about 0.57 millimeters thick. Each of tines 10 is beveled at a 60 degree angle, and each bevel 90 is about 0.869 millimeters in depth. FIG. 5 illustrates a preferred embodiment of a 1.5 millimeter condylar plate 40.

### Example 2

### 2.0 Millimeter Condylar Plate

The 2.0 millimeter condylar plate 40 is configured with five apertures 20, 30, all of which are configured to receive a 2.0 millimeter bone screw. Four of apertures 20 are substantially rounded and cup-shaped, each having a diameter defined by a Gage Ball having about a 4.10 millimeter diameter. Apertures 20, 30 are situated along longitudinal axis 70 in a substantially straight line as follows: There is a substantially round aperture 20 located at the top of plate 40 between tines 10 which is bendable to optimize fixation of plate 40 to the bone. Aperture 30 is located just below tines 10 and is oval shaped and has a length along longitudinal axis 70 of plate 40 of about 4.325 millimeters. Aperture 30 is configured to receive the 2.0 millimeter screw, but allows for about 1 millimeter of movement in longitudinal axis 70 of plate 40 to optimize the fixation of plate 40 on the bone. There are three substantially round apertures 20 that follow the oval-shaped aperture 30, which are spaced substantially equally apart at a distance of about 6.5 millimeters from center point to center point of each aperture 20.

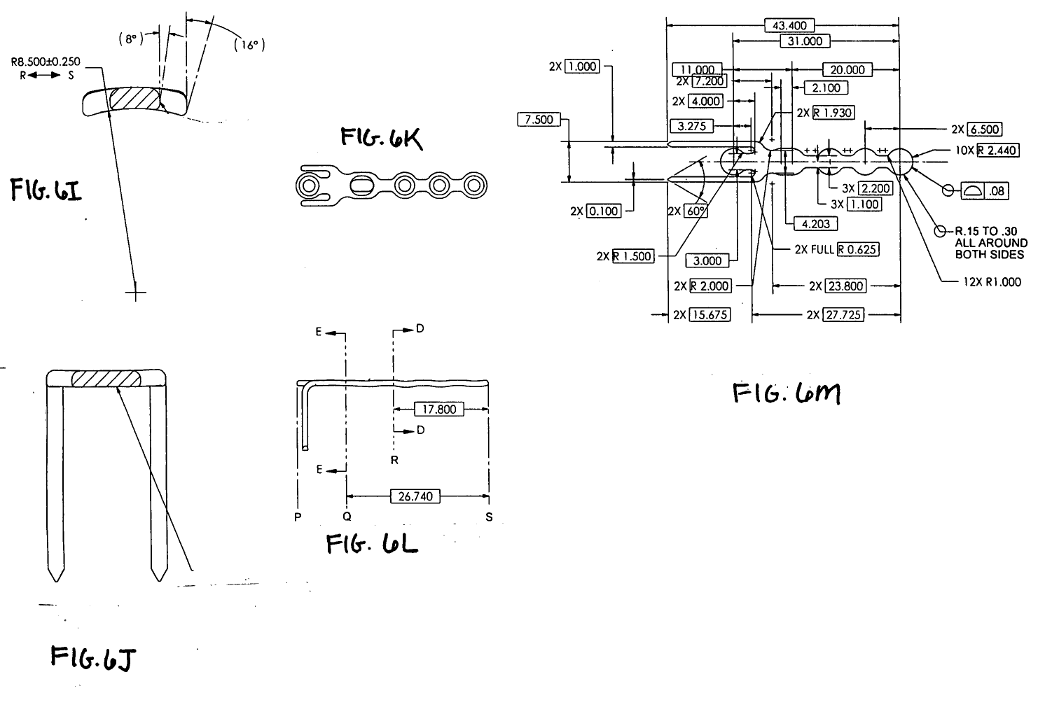

Plate 40 is about 35.880 millimeters long and about 1.0 millimeter thick. Tines 10 are about 13.0 millimeters long, taking into account the thickness of plate 40. Tines 10 are about 1.0 millimeter thick. Each of tines 10 is beveled at a 60 degree angle, and each bevel 90 is about 0.774 millimeters in depth. FIG. 6 illustrates a preferred embodiment of a 2.0 millimeter condylar plate 40.

### Example 3

### 2.4 Millimeter Condylar Plate

The 2.4 millimeter condylar plate 40 is configured with five apertures 20, 30, all of which are configured to receive a 2.4 millimeter bone screw. Four of the apertures 20 are substantially rounded and cup-shaped, each having a diameter defined by a Gage Ball having about a 4.50 millimeter diameter. Apertures 20, 30 are situated along longitudinal axis 70 in a substantially straight line as follows: There is a substantially round aperture 20 located at the top of plate 40 between tines 10 which is bendable to optimize fixation of plate 40 to the bone. Aperture 30 is located just below tines 10 and is oval shaped and has a length along longitudinal axis 70 of plate 40 of about 4.725 millimeters. Aperture 30 is configured to receive the 2.4 millimeter screw, but allows for about 1 millimeter of movement in longitudinal axis 70 of plate 40 to optimize the fixation of plate 40 on the bone. There are three substantially round apertures 20 that follow the oval-shaped aperture 30, which are spaced substantially equally apart at a distance of about 6.500 millimeters from center point to center point of each aperture 20.

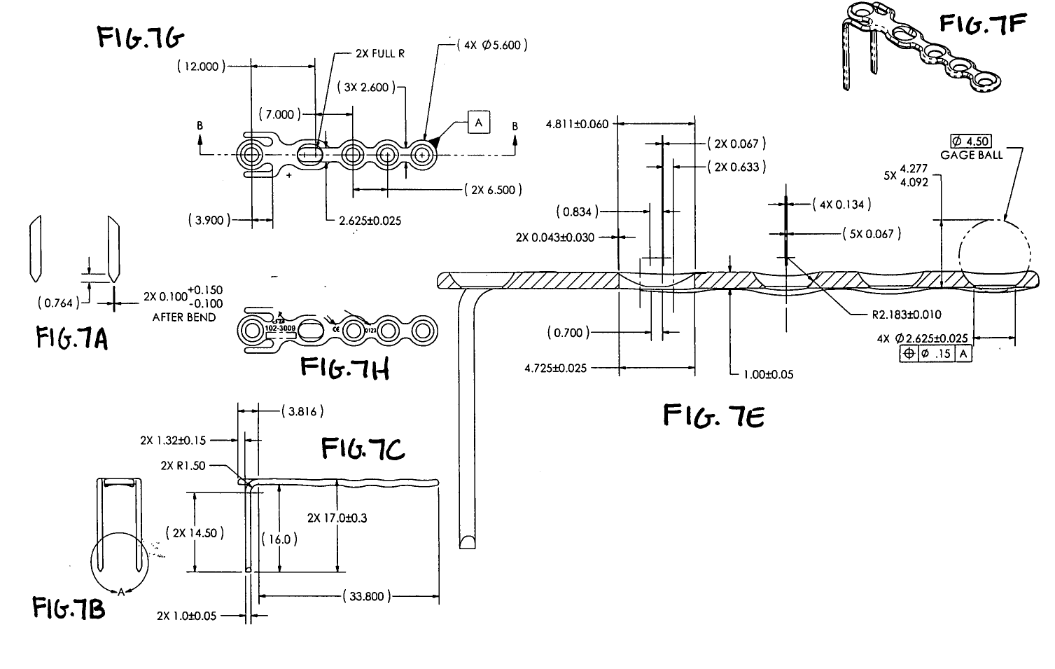

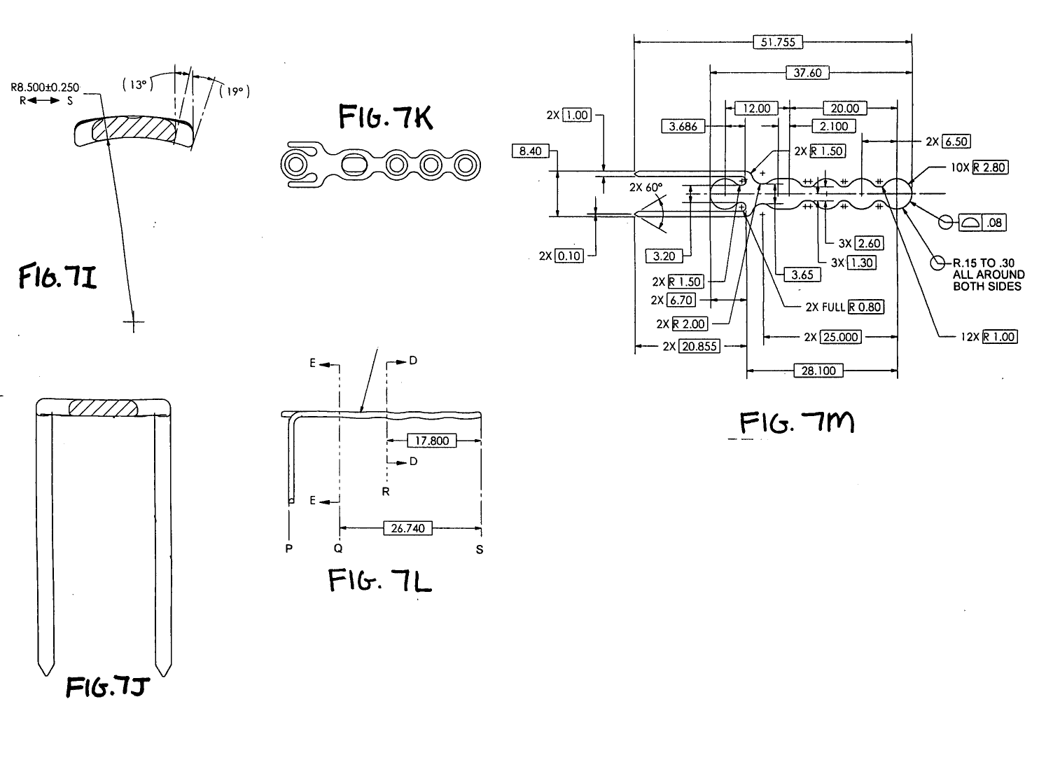

Plate 40 is about 37.616 millimeters long and about 1.00 millimeter thick. Tines 10 are about 17.0 millimeters long, taking into account the thickness of plate 40. Tines 10 are about 1.0 millimeter thick. Each of tines 10 is beveled at a 60 degree angle, and each bevel 90 is about 0.764 millimeter in depth. FIG. 7 illustrates a preferred embodiment of a 2.4 millimeter condylar plate 40.

It will be apparent to those skilled in the art that various modifications and variations can be made in the device of the present invention without departing from the scope of the invention. Thus, it is intended that the present invention cover the modifications and variations of the present invention provided they come within the scope of the appended claims and their equivalents.

What is claimed is:

1. A condylar plate for fixation of a small bone fracture comprising:
a. two or more tines; and
b. a plate integral with said tines and comprising one or more apertures configured to receive a fastener,
wherein said tines of said condylar plate are configured to be fixed to the condyle portion of said small bone.

2. The condylar plate of claim 1, wherein said plate comprises scalloped edges.

3. The condylar plate of claim 1, wherein said tines are normal to said plate.

4. The condylar plate of claim 1, wherein all or a portion of each of said tines is removable.

5. The condylar plate of claim 1, wherein said tines are flexible.

6. The condylar plate of claim 1, wherein said plate comprises five apertures.

7. The condylar plate of claim 1, wherein at least one of said apertures is substantially round.

8. The condylar plate of claim 1, wherein at least one of said apertures is substantially oval.

9. The condylar plate of claim 1, wherein said fasteners are selected from the group consisting of screws, k-wires, pins, and combinations thereof.

10. The condylar plate of claim 9, wherein said fasteners are screws.

11. The condylar plate of claim 1, wherein said fastener includes a head that is configured to be substantially flush with said plate.

12. A method of fitting a condylar plate to a small bone in a patient, said method comprising:
a. molding a pliable condylar plate template having a shape that corresponds to a shape of said condylar plate to said small bone, wherein said template comprises a longitudinal planar shaft substantially identical to a longitudinal planar shaft of the condylar plate, and wherein said template comprises apertures in a position substantially identical to a location of said tines are located in the condylar plate;
b. drilling holes through said apertures into said bone to indicate where the tines of the condylar plate will be fixated to said bone; and
c. bending said condylar plate to conform to said template configuration.
